(19) United States
(12) Patent Application Publication
(10) Pub. No.: US 2014/0156296 A1
(43) Pub. Date: Jun. 5, 2014

(54) Medication Adherence Scoring System and Method

(71) Applicant: 12th MAN TECHNOLOGIES, INC., Garden Grove, CA (US)

(72) Inventors: Alex Stenzler, Long Beach, CA (US); Steve Han, Huntington Beach, CA (US); Joan T. Waltman, Rancho Santa Fe, CA (US)

(73) Assignee: 12th MAN TECHNOLOGIES, INC., Garden Grove, CA (US)

(21) Appl. No.: 14/096,781

(22) Filed: Dec. 4, 2013

Related U.S. Application Data
(60) Provisional application No. 61/733,377, filed on Dec. 4, 2012.

(51) Int. Cl. A61B 5/00

(52) U.S. Cl. CPC A61B 5/4833
USPC 705/2

(57) ABSTRACT

A method of measuring a user's adherence to a medication schedule is described. The method includes the steps of tracking the time between medication doses, alerting the user when it is time to take a medication dose, tracking the elapsed time between the alert and when the user takes the medication dose, and comparing the elapsed time to a scoring metric to calculate a score, wherein the score is representative of the user's adherence to the medication schedule. An automated system for measuring a user's adherence to a medication schedule is also described. The system includes a computing device that executes a software application that includes a tracking mechanism that alerts the user when a next medication dose is to be taken and tracks the amount of elapsed time from the alert to the time that the user takes the medication dose, and a scoring mechanism that compares the amount of elapsed time to a scoring metric to determine a medication adherence score.

### CROSS-REFERENCE TO RELATED APPLICATIONS

This application claims the benefit of priority to U.S. Provisional Application Ser. No. 61/733,377, filed Dec. 4, 2012, the entire disclosure of which is incorporated by reference herein in its entirety.

### BACKGROUND OF THE INVENTION

A patient's adherence to medical therapies is vital to the overall success of prescribed or suggested therapy. Some estimates suggest that only about half of patients with chronic diseases follow treatment recommendations (World Health Organization, 2003, Adherence to Long-Term Therapies: Evidence for Action). Non-adherence can have obvious detrimental effects on the patient, but it also has harmful economic and societal impacts. For example, non-adherence can result in a patient requiring treatment for a longer than otherwise necessary period of time. Acknowledgment of the importance of medical adherence has led to the development of numerous types of strategies to improve patients' adherence.

Some strategies of improving medical adherence are focused on the actual therapy being suggested. Improving adherence can, in some instances, be improved by altering the types of treatments that are prescribed or recommended. For example, a health care provider may only recommend strategies that are not overly burdensome for the patient. Further, efforts at reducing side effects of medications and the development of longer active formulations may result in the patient remaining adherent to a particular medication.

Other strategies of improving adherence have focused on the development of adherence monitoring systems and/or systems which provide reminders to take proper medication. Some strategies have involved real time monitoring of when a patient actually ingests a medication. For example, a pill can activate a transmitter as it enters the stomach. However, the technology needed to make this strategy economically feasible for a wide range of patients is lacking. Other strategies have included pill boxes and pill dispensers that provide a patient with the right medication at the right time. While these strategies remain simple, they do little to actually motivate or induce a patient to remain adherent to their treatment regimen.

Thus, there is a need in the art for systems and methods to provide reminders and motivation to a patient in order to improve adherence to medication schedules. The present invention satisfies this unmet need.

### SUMMARY OF THE INVENTION

The present invention relates to systems and methods for measuring and improving a user's adherence to a medication schedule.

In one embodiment, the present invention is an automated system for measuring a user's adherence to a medication schedule, comprising: a computing device that executes a software application that includes a tracking mechanism and a scoring mechanism; wherein the tracking mechanism alerts the user when a next medication dose is to be taken and tracks the amount of elapsed time from the alert to the time that the user takes the medication dose; and wherein the scoring mechanism compares the amount of elapsed time to a scoring metric to determine a medication adherence score. In another embodiment, the software application of the system of the present invention is a hosted application accessible to the computing device over a communications network.

In one embodiment, the present invention is a method of measuring a user's adherence to a medication schedule, comprising: tracking the time between medication doses; alerting the user when it is time to take a medication dose; tracking the elapsed time between the alert and when the user takes the medication dose; and comparing the elapsed time to a scoring metric to calculate a score, wherein the score is representative of the user's adherence to the medication schedule. In another embodiment, the method of the present invention further comprises repeating the steps of alerting the user when it is time to take a medication dose, tracking the elapsed time between the alert and when the user takes the medication dose, and comparing the elapsed time to a scoring metric to calculate a score for a plurality of consecutively taken doses over a designated period of time, and calculating an average medication adherence score for the designated period of time.

In another embodiment, the present invention relates to a method of improving a user's adherence to a medication schedule composed of at least two designated time periods, comprising: tracking the time between medication doses within a first designated time period; alerting the user when it is time to take a medication dose during the first designated time period; tracking the elapsed time between the alert and when the user takes each medication dose during the first designated time period; comparing the elapsed times to a scoring metric to calculate a score, wherein the score is representative of the user's adherence to the medication schedule during the first designated time period of the medication schedule; and incentivizing the user to improve their score for a second designated time period of the medication schedule by assigning a grade that is representative of the user's adherence to the medication schedule during the first designated time period. In yet another embodiment, the methods of the present invention are executed by a software application resident on a computing device, or executed by a hosted application accessed by a computing device over a communications network.

In one embodiment, the medication adherence score of the systems or methods of the present invention is determined for a plurality of consecutively taken doses, and an average medication adherence score is calculated for the period comprising the taken doses. In one embodiment, the average medication adherence score is calculated based on the sum of each medication adherence score for each dose taken divided by the total number of doses taken in the period.

In one embodiment, the scoring metric of the present invention uses a score of 100 for an elapsed time of 0-60 minutes from the alert to the time that the user takes the medication dose, a score of 75 for an elapsed time of 61-120 minutes, a score of 50 for an elapsed time of 121-180 minutes, a score of 25 for an elapsed time of 181-240 minutes, and a score of 0 for an elapsed time of greater than or equal to 241 minutes. In another embodiment, the scoring metric of the present invention uses a letter grade of “A” for an average medication adherence score of greater than or equal to 90, as calculated in the description above, a letter grade of “B” for an average medication adherence score of between 80-89, a letter grade of “C” for an average medication adherence score of between 70-79, a letter grade of “D” for an average medication adherence score of between 50-69, and a letter grade of “F” for an average medication adherence score of below 50. In yet another embodiment, the systems or methods of the present invention further comprise a report that is representative of the user's performance in adhering to the medication schedule.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1, comprising FIGS. 1A and 1B, is a series of exemplary medication adherence score report formats. FIG. 1A is a pie chart of a user's medication adherence score, while FIG. 1B is a line graph of a user's medication adherence score.

FIG. 2 is a flow chart of an exemplary method of measuring a user's adherence to a medication schedule.

### DETAILED DESCRIPTION

It is to be understood that the figures and descriptions of the present invention have been simplified to illustrate elements that are relevant for a clear understanding of the present invention, while eliminating, for the purpose of clarity, many other elements found in medication adherence systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

The present invention relates to a medication reminder system to monitor and improve the user's adherence to taking their medications according to a prescribed dosage schedule assigned to each medication, respectively. The system is preferably operated at least in part through a software application executed on a computing device, such as a desktop, laptop, tablet, pad, smartphone or other computing or thin client device having a visual display. Further description of the system as an electronic software or hosted online platform is provided elsewhere herein.

For example, in one embodiment, the invention includes a medication tracking and scoring engine, operating as a fully automated software platform that can be executed by any stationary, portable, networked or stand-alone computing device. In other embodiments, the invention can be partially embodied within a software platform, while selected procedural steps are executed manually. In other embodiments, the invention can also be integrated into hospital system databases, or pharmacy databases, such that selected patient records or other information items within the hospital or pharmacy system can be collected and used in the tracking and scoring mechanisms, or can be used to populate and report to the system databases, all in a manner that is compliant with HIPAA regulations.

The system includes a data collection and/or data input mechanism to establish the parameters of a particular medication dosage schedule. Such input mechanism may include a data upload, or keyed entry of data via a keyboard, touchscreen or similar data entry mechanism. The system incorporates this data (also referred to herein as one or more information items) into both a medication schedule tracking mechanism and a scoring mechanism. The information items may be collected from or entered by the patient, a patient representative or proxy, doctor, pharmacist, a hospital or health care facility, a pharmacy or from a database of existing data or other records. For example, and without limitation, the information items may correspond to the type of medication, dosage, number of doses available, time interval between doses, warnings, side effects, and any patient information. It should be appreciated that other factors, as would be understood by those skilled in the art, may be incorporated into the collected information items without departing from the spirit of the present invention. It should also be appreciated that the present invention is not limited to any particular combination of factors, or weighting of factors, in the tracking and scoring of a user's ability to adhere to a medication schedule.

As contemplated herein, the tracking mechanism identifies the time interval between doses for a particular medication, and further identifies when a particular medication dose was (or should be) taken. In certain embodiments, the tracking mechanism may initiate an alert or alarm that signals that the next medication dose is to be taken, and/or it can signal a reminder that a dose should be taken in the near future, such within the next hour, 45 minutes, 30 minutes, 15 minutes, 10 minutes, 5 minutes, or any other desirable time period. In other embodiments, the tracking mechanism may include a “cancel” or “snooze” feature, whereby the user either cancels the tracking mechanism for a particular dose, or it can reset the alert or alarm to signal again at a later point in time, such as for 60 minutes later, 45 minutes later, 30 minutes later, 15 minutes later, 10 minutes later, 5 minutes later, or any other later time period desired. Further still, the tracking system may initiate a timer at the designated time to take a medication dose, such that the tracking mechanism calculates the amount of elapsed time it takes for the user to identify that the medication has in fact been taken. In certain embodiments, the tracking mechanism may include an override function, whereby a person may enter the elapsed time interval as an information item. For example, if a patient takes a medication dose at a time interval of 4 hours, but cannot access the tracking mechanism for an extra hour later, the patient may override the tracked 5 hour time interval and enter the time interval as 4 hours. Likewise, the patient may override the tracking mechanism during the subsequent dosage interval, by adding an hour to the time being tracked between doses, to reflect that the dose was taken an hour earlier.

As contemplated herein, the system may also include a scoring mechanism. The scoring mechanism is based on the results of the tracking mechanism, and provides a measurement of the user's adherence to the tracked medication schedule. The scoring mechanism utilizes a scoring algorithm that calculates a value representative of the user's ability to adhere to the prescribed medication schedule. The scoring metric, or algorithm, can optionally weight each information item category in the system, or any tracked time period, and calculate a value that is determinative of the tracking and scoring of the user's ability to adhere to a medication schedule. It should be appreciated that the values designated for each information item category may vary according to the target user and/or type of medication monitored. Further, the number or combination of information item categories will also effect the values designated. Further, the final score of the system may be set as a threshold value, where a score of equal to or above a designated value indicates a particular grade level achieved. Alternatively, final score ranges can be used to designate categories. As contemplated herein, the scoring output of the system is designed to motivate a user to improve their adherence score, and ultimately increase their ability to adhere to the prescribed medication schedule.

For example, the calculated value may take the form of a performance “grade,” such as a letter grade of A, B, C, D, or F, and/or as a number grade, such as a score between 0 and 100, as is commonly used in academic environments. In other embodiments, the calculated value may be presented as a performance rating, such as a rating of “excellent,” “good,” “satisfactory” or “poor.” It should be appreciated that the present invention may utilize any sort of grading or performance rating scale or format as would be understood by those skilled in the art.

Accordingly, in one exemplary embodiment, the system includes a computing device, such as a network enabled tablet computer executing a software application that includes the tracking and scoring mechanisms as described herein. The information relating to the medication the user is taking (such as one pill every four hours) is input into the system, and the tracking mechanism is initiated. At four hours, the system initiates an alert (audible alarm and/or digital message), signaling that it is time to take the pill. The computing device also initiates a timer that tracks the amount of time from the end of the four hour period. When the user has taken the pill, the user hits “cancel” or “taken” to stop the timer. The scoring mechanism then compares the timer value to a scoring metric, such as the scoring metric in Table 1, and assigns the requisite score.

TABLE 1

| Time elapsed from end of period | Score |
|---|---|
| 0-60 minutes | 100 |
| 61-120 minutes | 75 |
| 121-180 minutes | 50 |
| 181-240 minutes | 25 |
| 241+ minutes | 0 |

In the event that a patient takes a medication dose before the designated time (i.e., prior to the alert that it is time to take a dose), the scoring mechanism may instead calculate the time difference between the predetermined dosage interval and the actual (shortened) time interval taken. Accordingly, the scoring metric may reduce the overall score if a dose is taken too early.

The process is repeated for each four hour period until the end of the medication schedule, or until the end of a designated time period that can be different than the complete medication schedule, such as daily, weekly, monthly, or any other period desired. At the end of any such period, the scores are added up and the sum is divided by the total number of medication reminders sent to calculate an average score.

Depending on the scoring output desired, the numerical values can be further categorized into letter grades, such as according to the grading metric of Table 2.

TABLE 2

| Average score | Letter Grade |
|---|---|
| ≧90 | A |
| 80-89 | B |
| 70-79 | C |
| 50-69 | D |
| <50 | F |

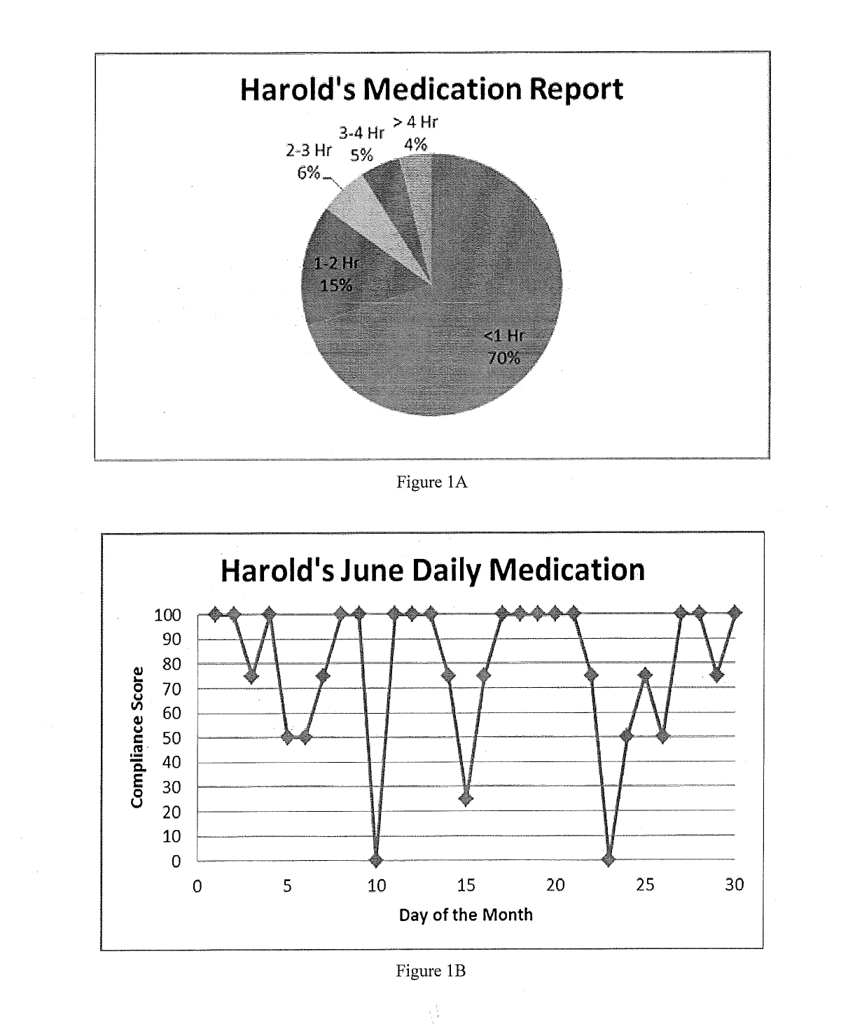

The system may optionally report the numerical or letter grade to the user or other interested party on the computing device visual display, or to a secondary device via an email or text message. Such a report may be a textual message, such as “Your Grade this week is a B,” and it may include any sort of emotionally positive or negative image, such as smiling face or a frowning face. Such reports may also include greater detail, such as a pie chart (FIG. 1A) or a line graph (FIG. 1B) of the user's performance. The reported grade is used to incentivize the user to obtain a better grade in the next designated time period.

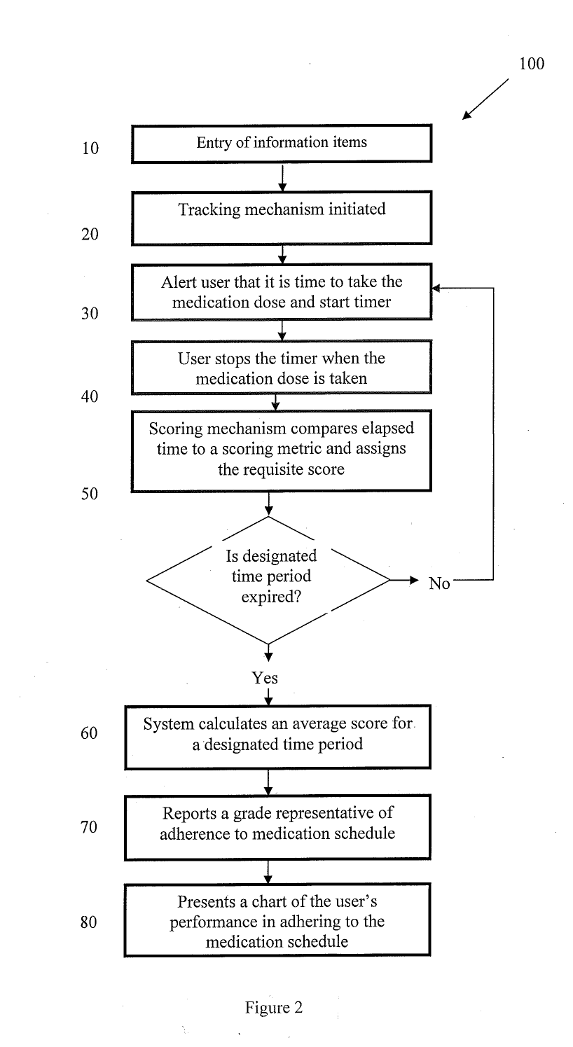

Accordingly, the present invention also includes a method of measuring and improving a user's adherence to a prescribed medication schedule using the aformentioned system. Referring now to FIG. 2, a flow chart of an exemplary method 100 of the present invention is illustrated. At step 10, at least one information item relating to the frequency of a medication dose schedule is entered into the aforementioned system. At step 20, the system tracking mechanism is initiated according to the at least one information item entered. At step 30, the system alerts the user that it is time to take the medication dose, and a timer tracks the amount of time that elapses from when the alert signal was sent. At step 40, the user stops the timer when the medication dose has been taken. At step 50, the system scoring mechanism compares the elapsed time from the timer to a scoring metric and assigns the requisite score. The process of steps 30, 40 and 50 are repeated for each dose for a designated period of time. At the end of the designated period of time, at step 60 the system calculates an average score for the designated time period, and at step 70, the system reports a grade to the user that is representative of their adherence to the medication schedule. At step 80, the system optionally presents a formatted report of the user's performance in adhering to the medication schedule.

As mentioned previously, the system of the present invention may operate on a computer platform, such as a local or remote executable software platform, or as a hosted internet or network program or portal. In certain embodiments, only portions of the system may be computer operated, or in other embodiments, the entire system may be computer operated. As contemplated herein, any “computer platform” may be operable from any computing device as would be understood by those skilled in the art, including desktop or mobile devices, laptops, desktops, tablets, smartphones or other wireless digital/cellular phones, televisions or other thin client devices.

For example, the computer operable component(s) of the system may reside entirely on a single computing device, or may reside on a central server and run on any number of end-user devices via a communications network. The computing devices may include at least one processor, standard input and output devices, as well as all hardware and software typically found on computing devices for storing data and running programs, and for sending and receiving data over a network, if needed. If a central server is used, it may be one server or, more preferably, a combination of scalable servers, providing functionality as a network mainframe server, a web server, a mail server and central database server, all maintained and managed by an administrator or operator of the system. The computing device(s) may also be connected directly or via a network to remote databases, such as for additional storage backup, and to allow for the communication of files, email, software, and any other data format between two or more computing devices. The communications network can be a wide area network and may be any suitable networked system understood by those having ordinary skill in the art, such as, for example, an open, wide area network (e.g., the internet), an electronic network, an optical network, a wireless network, a physically secure network or virtual private network, and any combinations thereof. The communications network may also include any intermediate nodes, such as gateways, routers, bridges, internet service provider networks, public-switched telephone networks, proxy servers, firewalls, and the like, such that the communications network may be suitable for the transmission of information items and other data throughout the system.

Further, the communications network may also use standard architecture and protocols as understood by those skilled in the art, such as, for example, a packet switched network for transporting information and packets in accordance with a standard transmission control protocol/Internet protocol (“TCP/IP”). Any of the computing devices may be communicatively connected into the communications network through, for example, a traditional telephone service connection using a conventional modem, an integrated services digital network (“ISDN”), a cable connection including a data over cable system interface specification (“DOCSIS”) cable modem, a digital subscriber line (“DSL”), a T1 line, or any other mechanism as understood by those skilled in the art. Additionally, the system may utilize any conventional operating platform or combination of platforms (Windows, Mac OS, Unix, Linux, Android, etc.) and may utilize any conventional networking and communications software as would be understood by those skilled in the art.

To protect data and to assist in complying with HIPAA regulations (when required), an encryption standard may be used to protect files from unauthorized interception over the network. Any encryption standard or authentication method as may be understood by those having ordinary skill in the art may be used at any point in the system of the present invention. For example, encryption may be accomplished by encrypting an output file by using a Secure Socket Layer (SSL) with dual key encryption. Additionally, the system may limit data manipulation, or information access. For example, a system administrator may allow for administration at one or more levels, such as at an individual user (patient) level, a healthcare professional level, or at a system level. A system administrator may also implement access or use restrictions for users at any level. Such restrictions may include, for example, the assignment of user names and passwords that allow the use of the present invention, or the selection of one or more data types that the subservient user is allowed to view or manipulate.

As mentioned previously, the system may operate as application software, which may be managed by a local or remote computing device. The software may include a software framework or architecture that optimizes ease of use of at least one existing software platform, and that may also extend the capabilities of at least one existing software platform. The application architecture may approximate the actual way users organize and manage electronic files, and thus may organize use activities in a natural, coherent manner while delivering use activities through a simple, consistent, and intuitive interface within each application and across applications. The architecture may also be reusable, providing plug-in capability to any number of applications, without extensive re-programming, which may enable parties outside of the system to create components that plug into the architecture. Thus, software or portals in the architecture may be extensible and new software or portals may be created for the architecture by any party.

The system software may provide, for example, applications, such as the aforementioned medication adherence program, accessible to one or more users to perform one or more functions. Such applications may be available at the same location as the user, or at a location remote from the user. Each application may provide a graphical user interface (GUI) for ease of interaction by the user with information resident in the system. A GUI may be specific to a user, set of users, or type of user, or may be the same for all users or a selected subset of users. The system software may also provide a master GUI set that allows a user to select or interact with GUIs of one or more other applications, or that allows a user to simultaneously access a variety of information otherwise available through any portion of the system.

The system software may also be a portal that provides, via the GUI, remote access to and from the system of the present invention. The software may include, for example, a network browser, as well as other standard applications. The software may also include the ability, either automatically based upon a user request in another application, or by a user request, to search, or otherwise retrieve particular data from one or more remote points, such as on the internet. The software may vary by user type, or may be available to only a certain user type, depending on the needs of the system. Users may have some portions, or all of the application software resident on a local computing device, or may simply have linking mechanisms, as understood by those skilled in the art, to link a computing device to the software running on a central server via the communications network, for example. As such, any device having, or having access to, the software may be capable of uploading, or downloading, any information item or data collection item, or informational files to be associated with such files.

Presentation of data through the software may be in any sort and number of selectable formats. For example, a multi-layer format may be used, wherein additional information is available by viewing successively lower layers of presented information. Such layers may be made available by the use of drop down menus, tabbed folder files, or other layering techniques understood by those skilled in the art. Formats may also include AutoFill functionality, wherein data may be filled responsively to the entry of partial data in a particular field by the user. All formats may be in standard readable formats, such as XML. The software may further incorporate standard features typically found in applications, such as, for example, a front or “main” page to present a user with various selectable options for use or organization of information item collection fields.

The system software may also include standard reporting mechanisms, such as generating a printable results report, or an electronic results report that can be transmitted to any communicatively connected computing device, such as a generated SMS message, email message or file attachment. Likewise, particular results of the aforementioned medication adherence program can trigger an alert signal, such as the generation of an alert email, text or phone call, to alert the user, a family member or guardian, an expert, clinician or other healthcare professional of the particular results.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. An automated system for measuring a user's adherence to a medication schedule, comprising:
a computing device that executes a software application that includes a tracking mechanism and a scoring mechanism;
wherein the tracking mechanism alerts the user when a next medication dose is to be taken and tracks the amount of elapsed time from the alert to the time that the user takes the medication dose; and
wherein the scoring mechanism compares the amount of elapsed time to a scoring metric to determine a medication adherence score.

2. The system of claim 1, wherein a medication adherence score is determined for a plurality of consecutively taken doses, and an average medication adherence score is calculated for the period comprising the taken doses.

3. The system of claim 2, wherein the average medication adherence score is calculated based on the sum of each medication adherence score for each dose taken divided by the total number of doses taken in the period.

4. The system of claim 3, wherein the scoring metric assigns a score of 100 for an elapsed time of 0-60 minutes, a score of 75 for an elapsed time of 61-120 minutes, a score of 50 for an elapsed time of 121-180 minutes, a score of 25 for an elapsed time of 181-240 minutes, and a score of 0 for an elapsed time of greater than or equal to 241 minutes.

5. The system of claim 4, wherein the scoring metric assigns a letter grade of “A” for an average medication adherence score of greater than or equal to 90, a letter grade of “B” for an average medication adherence score of between 80-89, a letter grade of “C” for an average medication adherence score of between 70-79, a letter grade of “D” for an average medication adherence score of between 50-69, and a letter grade of “F” for an average medication adherence score of below 50.

6. The system of claim 5, further comprising a report that is representative of the user's performance in adhering to the medication schedule.

7. The system of claim 1, wherein the software application is a hosted application accessible to the computing device over a communications network.

8. A method of measuring a user's adherence to a medication schedule, comprising:
tracking the time between medication doses;
alerting the user when it is time to take a medication dose;
tracking the elapsed time between the alert and when the user takes the medication dose; and
comparing the elapsed time to a scoring metric to calculate a score, wherein the score is representative of the user's adherence to the medication schedule.

9. The method of claim 8, further comprising repeating the steps of alerting the user when it is time to take a medication dose, tracking the elapsed time between the alert and when the user takes the medication dose, and comparing the elapsed time to a scoring metric to calculate a score for a plurality of consecutively taken doses over a designated period of time, and calculating an average medication adherence score for the designated period of time.

10. The method of claim 9, wherein the average medication adherence score is calculated based on the sum of each medication adherence score for each dose taken divided by the total number of doses taken in the designated period of time.

11. The method of claim 10, wherein the scoring metric assigns a score of 100 for an elapsed time of 0-60 minutes, a score of 75 for an elapsed time of 61-120 minutes, a score of 50 for an elapsed time of 121-180 minutes, a score of 25 for an elapsed time of 181-240 minutes, and a score of 0 for an elapsed time of greater than or equal to 241 minutes.

12. The method of claim 11, wherein the scoring metric assigns a letter grade of “A” for an average medication adherence score of greater than or equal to 90, a letter grade of “B” for an average medication adherence score of between 80-89, a letter grade of “C” for an average medication adherence score of between 70-79, a letter grade of “D” for an average medication adherence score of between 50-69, and a letter grade of “F” for an average medication adherence score of below 50.

13. The method of claim 12, further comprising providing a report that is representative of the user's performance in adhering to the medication schedule.

14. The method of claim 8, wherein the method is executed by a software application resident on a computing device or a hosted application accessed by a computing device over a communications network.

15. A method of improving a user's adherence to a medication schedule composed of at least two designated time periods, comprising:
tracking the time between medication doses within a first designated time period;
alerting the user when it is time to take a medication dose within the first designated time period;
tracking the elapsed time between the alert and when the user takes each medication dose within the first designated time period;
comparing the elapsed times to a scoring metric to calculate a score, wherein the score is representative of the user's adherence to the medication schedule during the first designated time period of the medication schedule; and
incentivizing the user to improve their score for a second designated time period of the medication schedule by assigning a grade that is representative of the user's adherence to the medication schedule during the first designated time period.
